(19) United States
(12) Patent Application Publication
(10) Pub. No.: US 2026/0035644 A1
(43) Pub. Date: Feb. 5, 2026

(54) ATMOSPHERICALLY SELF-CONTAINED MULTI-WELL PLATE

(71) Applicant: KING ABDULAZIZ UNIVERSITY, Jeddah (SA)

(72) Inventors: Majed Abdulsamad FELEMBAN, Jeddah (SA); Moaaz Asif D. MALIK, Jeddah (SA); Hossam Hatem TAYEB, Jeddah (SA); Raed Ismail FELIMBAN, Jeddah (SA); Adeel GULZAR CHAUDHARY, Jeddah (SA); Rahmah Abdulhadi ALSILMI, Jeddah (SA); Khalidah Khalid NASSER, Jeddah (SA)

(73) Assignee: KING ABDULAZIZ UNIVERSITY, Jeddah (SA)

(21) Appl. No.: 18/789,357

(22) Filed: Jul. 30, 2024

(51) Int. Cl. C12M 1/32; C12M 1/00

(52) U.S. Cl. CPC C12M 23/12; C12M 23/38; C12M 29/00

(57) ABSTRACT

A multi-well plate includes a base and a lid. The base includes multiple wells arranged in a rectangular array, each well having a cylindrical shape with a circular opening on a top end of the well. A perimeter wall is arranged about a periphery of the rectangular array and includes a stacked edge arranged about an entirety of the perimeter wall. A gasket is attached to the perimeter wall at a notch on the stacked edge of the perimeter wall. The lid includes multiple circular structure axially aligned with the plurality of wells of the base. Each circular structure includes a concentric circular aperture and an injection port disposed at a center of each concentric circular aperture on each of the plurality of circular structures. Each injection port includes a sealing septum, where the lid rests on the gasket.

### STATEMENT OF ACKNOWLEDGEMENT

Support provided by the Deputyship for Research & Innovation, Ministry of Education in Saudi Arabia, including funding this research work through the Institutional Fund Projects, Ministry of Education under grant number IFPRC-203-142-2020 and to King Abdulaziz University, DSR, Jeddah, Saudi Arabia is gratefully acknowledged.

### BACKGROUND

### Technical Field

The present disclosure is directed to cell culture plates for biological sampling, more particularly, directed to an atmospherically self-contained multi-well plate.

### Description of Related Art

The “background” description provided herein is to present the context of the disclosure generally. Work of the presently named inventors, to the extent it is described in this background section, as well as aspects of the description that may not otherwise qualify as prior art at the time of filing, are neither expressly nor impliedly admitted as prior art against the present invention.

Multi-well plates, also referred to as microtiter plates or microplates, are used in a wide variety of tasks and activities in medical, chemical, and biological laboratories. Multi-well plates have a plurality of sample wells typically arranged as a rectangular matrix or array. Typical multi-well plates use a 2:3 width to length ratio for the size of the matrix or array. For example, a 6-well plate has a 2×3 rectangular matrix having two rows with three wells in each row. Other examples of arrangements for a multi-well plate include 24 wells arranged in a 4×6 matrix, 96 wells arranged in an 8×12 matrix, and 384 wells arranged in a 16×24 array. These arrangements preserve the 2:3 width to length ratio. Some configurations do not use a 2:3 width to length ratio and instead use a 3:4 width to length ratio, such as plates having 12 wells arranged in a 3×4 matrix and 48 wells arranged in a 6×8 matrix. Some multi-well plates maintain similar exterior dimensions across different types of plates or numbers of wells. Some multi-well plates increase the number of wells by decreasing the size of each well. This allows for the plate to keep the same overall exterior dimensions to allow plates with different numbers of wells to be stackable. For example, a 24-well plate and a 96-well plate could have similar exterior dimensions such that when stacked, the stack has a uniform length and width. This may be advantageous for creating a secure or stable stack of plates.

In general, a multi-well plate includes two main components. Firstly, a lower plate including one or more cavities into which living biomatter can be inoculated along with a corresponding growth media. Further, a second part of a culture plate is a lid for the lower plate which serves as a physical barrier to entry of contaminants such as dust, airborne flora, and fauna.

In an exemplary biological laboratory setting, multi-well plates are used for the seeding, growth, replication, proliferation, and expansion of living cells in a controlled environment. The growth of these cells may either be specified or general in nature depending on growth conditions and a source of initial biological samples used for primary inoculation. Traditionally, various reagents and solvents have been utilised in specific quantities and specific conditions. However, certain types of cells grow under specific conditions requiring differing gaseous growth conditions such as a specific proportion of carbon dioxide in their immediate environment or a reduction of oxygen in their atmospheric environment. Such conditions may vary substantially from the overall environment found in everyday life, such as, but are not limited to, temperatures in the 20° C. to 35° C. range and the typical atmospheric concentrations of gases found in the environment. The substantial range and variation of culture conditions for cell types means that whilst in general multi-well plates are suitable for a broad variety of cell culture, they are inadequate as currently designed for specific niche cases. Hence, there arises a need for a specific requirement, sealed multi-well plate. Currently, there is no commercial equivalent available which offers the customisations necessary for sealed growth in a multi-well plate while still allowing the addition or removal of solvents, substances, fluids and or growth additives that are necessary in a case-by-case basis for the culturing and proliferation of specific cell types. Presently, a conventional culture plate must be housed in a separate enclosure that is capable of providing custom environmental conditions such as temperature and or gas levels. However, even such equipment has limitations in its ability to adjust the local environment to just one set of conditions. Replicating multiple varying atmospheric environmental conditions requires multiple incubators which are prohibitively expensive.

US20160250632A1 describes a lid for a multi-well plate having apertures, each aperture having a gas permeable membrane, and fittings extending from the bottom surface of the lid to fit with a multi-well plate to reduce evaporation of liquid contents from within the wells of the multi-well plate. Further, the lid may protect the contents of a multi-well plate from spilling or from mingling with the contents of a neighbouring well in the multi-well plate. The gas permeable membrane includes one or more of a continuous or discontinuous thin film associated with each aperture, a locally thinned portion of the main body, and a quantity of material at least partially filling each aperture. However, because the aperture has a gas permeable membrane, the lid is successful at preventing solid contamination, but it does not create a sealed atmosphere. That is, the lid creates a multi-well plate that is subject to the gases of the ambient atmosphere. Additionally, each of the plurality of wells in the multi-well plate does not appear to be accessed individually without removing the lid. This removal may allow solid contaminants to be introduced.

US20220073851A1 describes a system and a method for propagating cells. The system includes a multi well plate and a plate sealing means for sealing at least one well of the multi well plate. The plate sealing means includes at least one solid bulge or at least one bulge including a solid base, said at least one bulge consisting of a resilient elastomer to securely seal at least one well of the multi well plate. However, the multi well plate described in this reference has each individual well sealed on a top of the well. That is, each well has its own sealed atmosphere. There is no shared headspace common to multiple wells and no gas exchange can occur between wells.

US20200131459A1 describes microplate covers that seal microplates and the wells within microplates to retain or control desired environmental conditions within the test environment, such as moisture level, oxygen content, and the like. In some embodiments, the visibility and light transmission, refractive, and reflective properties through and from the tray covers may be controlled to facilitate sensing of tray contents via optical method. However, this reference does not appear to describe the possibility of manipulation of cell cultures present inside the microplate without removing the lid.

Accordingly, it is one object of the present disclosure is to provide a multi-well plate that may circumvent the aforementioned drawbacks such as inability of manipulation of a particular cell culture without affecting neighboring cell cultures, contaminant free manipulation, complete atmospheric isolation of wells in the plate, and economic cost of the multi-well plate for niche cases.

### SUMMARY

In an exemplary embodiment, a multi-well plate is described. The multi-well plate includes a base. The base includes a plurality of wells arranged in a rectangular array, each well having a cylindrical shape with a circular opening on a top end of the well. A perimeter wall is arranged about a periphery of the rectangular array and includes a stacked edge arranged about an entirety of the perimeter wall. A gasket is attached circumferentially to the perimeter wall at a notch on the stacked edge of the perimeter wall. The multi-well plate includes a lid including a plurality of circular structures axially aligned with the plurality of wells of the base. Each circular structure includes a concentric circular aperture and an injection port disposed at a center of each concentric circular aperture on each of the plurality of circular structures forming a plurality of injection ports. Each injection port includes a sealing septum, where the lid is configured to rest on the gasket attached circumferentially to the perimeter wall and thereby forming a seal about a periphery of the rectangular array.

In some embodiments, the seal formed about the periphery of the rectangular array prevents mixing of an internal atmosphere defined within the base and lid with an external atmosphere.

In some embodiments, each well in the rectangular array has a corresponding circular projection in the lid.

In some embodiments, the lid is configured to rest on the gasket such that a circular structure of the plurality of circular structures does not prevent gas exchange between a well and another well.

In some embodiments, the plurality of circular structures is arranged to form a regular rectangular grid.

In some embodiments, each sealing septum of the lid is formed of an elastomeric material and has a hollow frustoconical shape with a narrow end, a wide end, and a sloping wall circumferentially connecting an edge of the narrow end and an edge of the wide end. Both the edge of the narrow end and the edge of the wide end are circular, the narrow end is closed, and the wide end is open. A height of the sealing septum measured perpendicularly from a plane defined by the narrow end to a plane defined by the edge of the wide end is less than a diameter of the narrow end.

In some embodiments, the elastomeric material is a rubber.

In some embodiments, the gasket is formed of an elastomeric material.

In some embodiments, the elastomeric material of the gasket is a rubber.

In some embodiments, a ratio of a diameter of the circular opening on the top end of the well to a diameter of the concentric circular aperture is 1:1 to 10:1.

In some embodiments, the multi-well plate further includes a gas inlet disposed on the lid and includes an injection port including a sealing septum.

In some embodiments, the gas inlet is disposed on a top of the lid.

In some embodiments, the gas inlet is disposed on a top of the lid such that the gas inlet is not disposed above a corresponding well of the plurality of wells.

In some embodiments, the injection port of the gas inlet is formed of an elastomeric material.

In some embodiments, the multi-well plate further includes a lid retention device configured to securely attach the lid to the base.

In some embodiments, the gasket has a width of 1.0 to 2.5 mm.

In some embodiments, the wells are arranged into an array having a width to length ratio of 2:3.

In some embodiments, each well has a diameter of 15 to 22.5 mm.

In some embodiments, each concentric circular aperture has a diameter of 2.5 to 15 mm.

In some embodiments, each sealing septum has an upper retention flange having a diameter of 8.5 to 18.5 mm.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof may be readily obtained as the same becomes better understood by reference to the following detailed description when considered in connection with the accompanying drawings, wherein:

FIG. 1 is a schematic perspective view of a multi-well plate, according to certain embodiments.

FIG. 2A is a schematic top view of a base of the multi-well plate, according to certain embodiments.

FIG. 2B is a schematic side view of the base of the multi-well plate, according to certain embodiments.

FIG. 3A is a schematic top view of a lid of the multi-well plate, according to certain embodiments.

FIG. 3B is a schematic top view of the lid depicting a plurality of concentric circular apertures, according to certain embodiments.

FIG. 3C is a schematic perspective view depicting a sealing septum attached to the lid, according to certain embodiments.

FIG. 3D is a schematic perspective view of the sealing septum of FIG. 3C, according to certain embodiments.

FIG. 3E is a schematic side view of the lid of the multi-well plate, according to certain embodiments.

FIG. 4A is an exploded view of the multi-well plate, according to certain embodiments.

FIG. 4B is a schematic side view depicting an assembled multi-well plate, according to certain embodiments.

FIG. 5 is a schematic side view of the multi-well plate depicting a lid retention device, according to certain embodiments.

FIG. 6A is a schematic side view of the multi-well plate depicting a gas inlet on a side of the lid, according to certain embodiments.

FIG. 6B is a schematic top view of the multi-well plate depicting a gas inlet on a top of the lid, according to certain embodiments.

FIG. 7 is a schematic side view of a sealing septum having a bottom flange attached to the lid, according to certain embodiments.

### DETAILED DESCRIPTION

In the drawings, reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

The aspects of the present disclosure are directed to a multi-well plate. The multi-well plate includes a base and a lid. In some embodiments, the base includes a plurality of wells. Each well can be configured to hold contents, such as solids, liquids, gels, or combinations of these. In some embodiments, the lid incorporates features specific to a purpose of isolating the contents of one or more of the plurality of wells in the base of the multi-well plate from contact and exposure (direct and indirect) to the external environment. In some embodiments, the lid includes a plurality of injection ports.

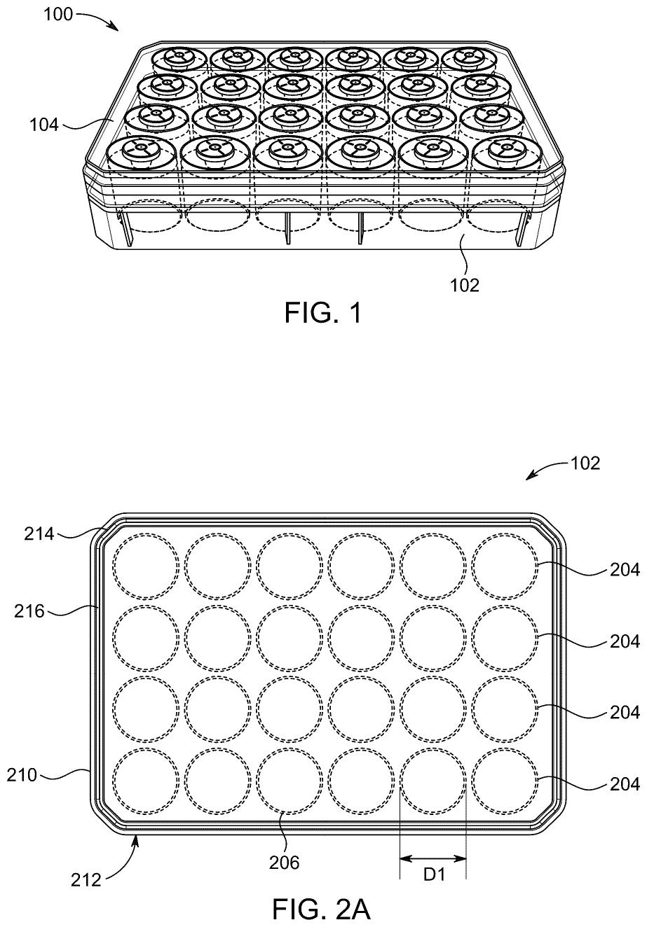

Referring to FIG. 1, a schematic perspective view of a multi-well plate 100 is illustrated, according to certain embodiments. Typically, multi-well plates are used in laboratories in order to provide a controlled environment for activities such as the seeding, growth, replication, proliferation, and expansion of viruses and living cells, such as, bacteria, fungi, single celled organisms, animal cells and tissues, and the like. Multi-well plates may also be used for experiments involving viruses or living cells or other experiments which can conveniently be performed in bulk or in multiple replicates using the plurality of wells. As depicted in FIG. 1, the multi-well plate 100 includes a base 102 and a lid 104 configured to be attached with a top end of the base 102. In particular, the base 102 is a structure configured to define multiple wells therein and the lid 104 is configured to close the wells to provide the controlled environment for culturing cells. In FIG. 1, the gasket described below has been omitted for clarity.

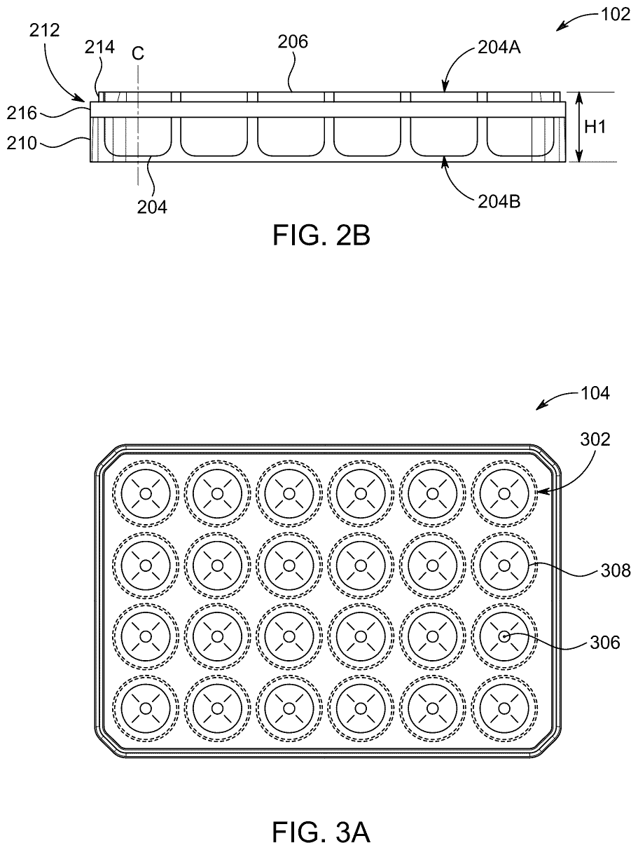

Referring to FIG. 2A and FIG. 2B, a schematic top view and a schematic side view, respectively, of the base 102 of the multi-well plate 100 is illustrated, according to certain embodiments. The exemplary base 102 depicted in FIGS. 2A and 2B includes a plurality of wells 204 arranged in a rectangular array. In general, the plurality of wells can be arranged in any suitable way. For example, the wells can be arranged irregularly. In some embodiments, the wells are arranged in an ordered array. In some embodiments, array can be a circular array, a square array, a rhomboidal array, a polygon shape array, or a combination thereof. In some embodiments, the array can have a uniform spacing between the wells. Such an array with a consistent arrangement and uniform spacing can be referred to as a “regular array”. In some embodiments, the array can have a non-uniform spacing between the wells. The number of the wells 204 included in the multi-well plate 100 may depend on an intended application or use of the multi-well plate. In general, the multi-well plate of the present disclosure can include any suitable number of wells. For example, the multi-well plate can have 2 wells, 3 wells, 4 wells, 6 wells, 8 wells, 10 wells, 12 wells, 14 wells, 15 wells, 16 wells, 18 wells, 20 wells, 21 wells, 22 wells, 24 wells, 27 wells, 28 wells, 30 wells, 32 wells, 34 wells, 36 wells, 39 wells, 40 wells, 42 wells, 44 wells, 45 wells, 46 wells, 48 wells, 49 wells, 50 wells, 56 wells, 96 wells, 384 wells, or 1536 wells. In some embodiments, the wells are arranged in an array having rows and columns. In some embodiments, the wells are arranged into an array having a width to length ratio of 2:3. For example, a 6-well plate may have a 2×3 rectangular matrix having two rows with three wells in each row. Other examples of arrangements for a multi-well plate include 24 wells arranged in a 4×6 matrix, 96 wells arranged in an 8×12 matrix, and 384 wells arranged in a 16×24 array. In some embodiments, the wells are arranged into an array having a width to length ratio that is not 2:3. For example, a multi-well plate can use a 3:4 width to length ratio. For example, a multi-well plate may have 12 wells arranged in a 3×4 matrix or 48 wells arranged in a 6×8 matrix. In some embodiments, the multi-well plate 100 includes twenty-four wells arranged in a 4×6 array. The 4×6 array may be defined as a configuration of the multi-well plate 100 having four rows and six columns of wells 204 or six rows and four columns of wells 204, which amounts to the total of twenty four wells. In other words, the 4×6 array may be defined as a size of the multi-well plate 100 having a width to length ratio of 4:6 (2:3 in reduced form). In some embodiments, the multi-well plate 100 may have a configuration of 1×2 array, 2×3 array, 4×6 array, 6×8 array, and the like.

In some embodiments, each well of the plurality of wells 204 has a cylindrical shape. The cylindrical shape can have a circular cross-section. The cylindrical shape can have straight walls and a constant diameter cross-section. The cylindrical shape can have sloping or curving walls and a non-constant diameter cross-section. In some embodiments, each well of the plurality of wells 204 has a shape having an elliptical cross-section. In some embodiments, each well of the plurality of wells 204 has a shape having a polygonal cross-section. For example, the well can have a cross-section that is a square, a rectangle, a rhombus, a hexagon, or some other polygon. In general, the polygon can be a regular polygon or an irregular polygon. In general, the wells having an elliptical cross-section or polygonal cross-section can have straight walls and a constant size cross-section or can have sloping or curving walls and a non-constant size cross-section. In some embodiments, the wells can have a circular opening 206 on a top end 204A of the well 204. That is, the top of the well is open. The open top can be totally open, that is, having an open top that is the same size as the well. Preferably, a bottom end 204B of each of the plurality of wells 204 is closed and supported by the base 102. In general, the bottom end 204B can be flat, curved, sloping, pointed, or some other shape. For example, a bottom end 204B can be flat, that is, the well has a constant depth or height. In some embodiments, the bottom end 204B can be pointed or curved, that is, the well does not have a constant depth or height. In some embodiments, the pointed or curved shape of the bottom end 204B can be shaped such that the well has a maximum depth or height in a center of the well. In some embodiments, the multi-well plate includes twenty-four wells. In some such embodiments, each well 204 of the twenty-four wells has a diameter ‘D1’ of 15 millimeters (mm) to 22.5 mm, preferably 15.5 to 21.5 mm, preferably 16 to 20.5 mm, preferably 16.5 to 19.5 mm, preferably 17 to 19 mm, preferably 17.5 to 18.5 mm, preferably 17.75 to 18.25 mm, preferably 18 mm. In some embodiments, the diameter of the circular opening 206 is equal to the diameter ‘D1’ of the well 204.

In some embodiments, the base is formed of a rigid material. Examples of suitable rigid materials include but are not limited to metals such as aluminum and stainless steel, glass, ceramic, and a polymer material such as a polystyrene, a polymethylpentene, a polypropylene, a polyethylene, a polytetrafluoroethylene, a polycarbonate, a polyacrylate, and combinations thereof.

In some embodiments, the base 102 of the multi-well plate 100 further comprises a perimeter wall 210 arranged about a periphery of the wells. In some embodiments where the wells are arranged in an array, the perimeter wall is arranged about a periphery of the array. For example, when the wells are arranged in a rectangular array, the perimeter wall can be disposed about a periphery of the rectangular array. It should be understood that the perimeter wall being arranged about a periphery of the wells means that the perimeter wall extends about a totality of the plurality of wells. In some embodiments, the perimeter wall can be disposed on or about a perimeter of the base 102. In some embodiments, the perimeter wall can be disposed on the base 102 at a location that is within the perimeter of the base 102 but still encompasses all of the wells. In some embodiments, the perimeter wall 210 is designed to extend upwards, in a vertical direction, from a bottom end of the base 102 of the multi-well plate 100. In other words, the perimeter wall 210 defines a height ‘H1’, such that a magnitude of the height ‘H1’ of the perimeter wall 210 is greater than or equal to a height of each of the plurality of wells 204. In some embodiments, the perimeter wall 210 defines a rectangular area including the plurality of wells 204 and space therebetween. The rectangular area may vary in accordance with the configuration of the multi-well plate 100, as such, a larger area is required to house a larger matrix of the plurality of wells 204. In some embodiments, the base 102 of the multi-well plate 100, in a twenty-four well configuration, has a length of about 127.5 mm, the height ‘H1’ of about 18.8 mm, and a width of about 85.3 mm.

In some embodiments, the perimeter wall includes a stacked edge 212. In some embodiments, the stacked edge 212 is arranged about a portion of the perimeter wall 210. In some embodiment, the stacked edge 212 is arranged about an entirety of the perimeter wall 210. The stacked edge 212 may be useful in disposing the lid on or attaching the lid to the base. In some embodiments, the stacked edge is oriented such that a lower portion of the stacked edge is oriented away from a center of the base and a raised portion of the stacked edge is oriented toward the center of the base. In some embodiments, the stacked edge is oriented such that a lower portion of the stacked edge is oriented toward a center of the base and a raised portion of the stacked edge is oriented away from the center of the base. In some embodiments, the lid or a portion thereof may rest on, be disposed on, or be attached to a lower portion of the stacked edge such that the raised portion of the stacked edge prevents the lid from moving laterally (e.g., sliding or slipping) off of the base. It should be understood that the lid or a portion thereof resting on, being disposed on, or being attached to the lower portion of the stacked edge does not preclude the lid or any portion thereof from resting on, being disposed on, or being attached to the raised portion of the stacked edge or any part of the perimeter wall or base. The lid or any portion thereof may rest on, be disposed on, or be attached to the raised portion of the stacked edge, the perimeter wall or any portion thereof, or any part of the base. In some embodiments, the stacked edge 212 may refer to a chamfered edge of the perimeter wall 210. In general, chamfering is a machining process to produce a transitional edge, which can also be referred to as the stacked edge 212.

In some embodiments, the multi-well plate 100 includes a gasket 216 attached to or disposed upon the perimeter wall 210. In some embodiments, the gasket 216 is attached to or disposed upon the stacked edge 212. In some embodiments, the gasket is attached to or disposed upon the lower portion of the stacked edge. In some embodiments, the stacked edge includes a notch 214. The notch can be formed in the lower portion of the stacked edge, the raised portion of the stacked edge, or both. The exemplary multi-well plate 100 depicted in FIG. 2B includes a gasket 216 attached to the perimeter wall 210 at the notch 214 on the stacked edge 212 of the perimeter wall 210. The stacked edge 212 and/or the notch 214 can allow for a better and secure fitment of the gasket 216 thereon. That is, the gasket 216 can securely fit into the notch 214.

In some embodiments, the gasket 216 is configured to provide a seal between the base 102 and the lid 104 to prevent mixing of an internal atmosphere defined between the base 102 and the lid 104 with an external atmosphere. That is, the seal may be gas tight. In some embodiments, the seal may be capable of withstanding a pressure differential between the internal atmosphere and external atmosphere of 0.001 to 1 atm, preferably 0.005 to 0.9 atm, preferably 0.01 to 0.75 atm, preferably 0.05 to 0.6 atm. In some embodiments, the gasket 216 is formed of an elastomeric material. Examples of elastomeric materials include but are not limited to rubbers such as natural rubber, nitrile rubber, butyl rubber, styrene-butadiene rubber, EPDM rubber, chloroprene, Neoprene®, hydrogenated nitrile rubber, and the like, silicones such as dimethylsilicone, methyl-phenylsilicone, methylvinylsilicone, mixtures thereof, and the like, fluoroelastomers such as FKM, FFKM, FEPM, and the like. In some embodiments, the elastomeric material is a rubber. In some embodiments, the gasket 216 has a width of 1.0 mm to 2.5 mm. In some embodiments, the gasket 216, attached to the perimeter wall 210, has a height of about 4 mm.

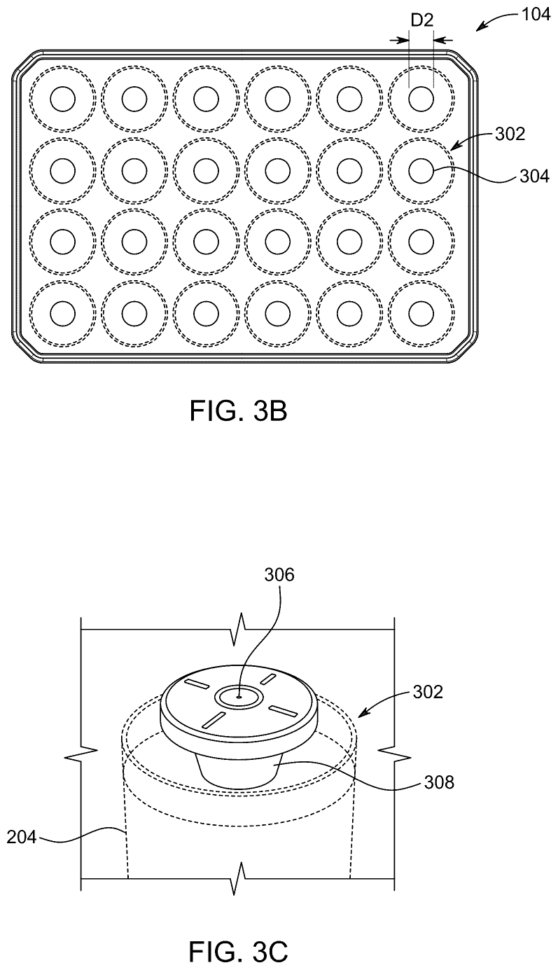

Referring to FIG. 3A, a schematic top view of the lid 104 of the multi-well plate 100 is illustrated, according to certain embodiments. The lid 104 also includes a plurality of structures. In general, the structures can have a shape which matches the cross-sectional shape of the wells in the base. In some embodiments, the structures are circular regardless of the cross-sectional shape of the wells in the base. In some embodiments, the lid 104 includes a plurality of circular structures 302 axially aligned with the plurality of wells 204 of the base 102. As such, a central axis ‘C’ (shown in FIG. 2B) of each well of the plurality of wells 204 aligns with a central axis ‘P’ (shown in FIG. 3E) of each circular structure of the plurality of circular structures 302. In some embodiments, each well of the plurality of wells 204 has a corresponding circular projection in the lid 104. In some embodiments, the plurality of circular structures forms an array which matches the array formed by the plurality of wells. That is, when the wells are arranged in an equally spaced array where each of the wells is equidistance from each other, each of the plurality of circular structures 302 is configured to be arranged at an equidistant interval from each other as well. Similarly, in embodiments where the wells for a rectangular array, the plurality of circular structures 302 is arranged to form a regular rectangular grid in the lid 104. In some embodiments, each well has only a single circular projection disposed above it. In some embodiments, each circular projection is disposed above only a single well. Preferably, each circular structure 302 has a diameter approximately equal to the diameter of a corresponding well. In some embodiments, the circular structures have a diameter of 15 to 22.5 mm, preferably 15.5 to 21.5 mm, preferably 16 to 20.5 mm, preferably 16.5 to 19.5 mm, preferably 17 to 19 mm, preferably 17.5 to 18.5 mm, preferably 17.75 to 18.25 mm, preferably 18 mm. In some embodiments, the lid is formed of a rigid material. Examples of suitable rigid materials include but are not limited to metals such as aluminum and stainless steel, glass, ceramic, and a polymer material such as a polystyrene, a polymethylpentene, a polypropylene, a polyethylene, a polytetrafluoroethylene, a polycarbonate, a polyacrylate, and combinations thereof. In some embodiments, the lid is formed of the same rigid material as the base. In some embodiments, the lid is formed of a different rigid material from the base.

As shown in FIG. 3B, each of the plurality of circular structures 302 includes a concentric aperture 304. In some embodiments, each concentric aperture 304 has an elliptical shape. In some embodiments, each concentric aperture 304 has a polygonal shape. For example, the concentric aperture 304 can have a shape that is a square, a rectangle, a rhombus, a hexagon, or some other polygon. In general, the polygon can be a regular polygon or an irregular polygon. In some embodiments, each concentric aperture 304 has a circular shape. Such a concentric aperture having a circular shape can be referred to as a “concentric circular aperture”. In some embodiments, the concentric circular aperture 304 has the diameter ‘D2’ of 2.5 to 15 mm, preferably 3 to 13 mm, preferably 3.5 to 11 mm, preferably 4 to 10 preferably 4.5 to 9.5 mm, preferably 5 to 9 mm, preferably 5.5 to 8.5 mm, preferably 6 to 8 mm, preferably 6.5 to 7.5 mm, preferably 6.75 to 7.25 mm, preferably 7 mm. The exemplary embodiment depicted in FIG. 3B, the concentric circular aperture 304 has a diameter ‘D2’ of 7 mm. In some embodiments, a ratio of a diameter of the circular opening 206 on the top end 204A of the well 204 to the diameter of the concentric circular aperture 304 is 1:1 to 10:1, preferably 1.25:1 to 7.5:1, preferably 1.5:1 to 5.0:1, preferably 1.75:1 to 4.0:1, preferably 2.0:1 to 3.5:1, preferably 2.25:1 to 3.0:1, preferably 2.5:1 to 2.75:1.

As shown in FIG. 3C, each of the plurality of circular structures 302 further includes an injection port 306 disposed at a center of each concentric circular aperture 304 on each of the plurality of circular structures 302 forming a plurality of injection ports 306. In some embodiments, each injection port 306 has a diameter of 0.5 mm to 13 mm, preferably 1 to 11 mm, preferably 1.5 to 9 mm, preferably 2 to 8 preferably 2.5 to 7.5 mm, preferably 3 to 7 mm, preferably 3.5 to 6.5 mm, preferably 4 to 6 mm, preferably 4.5 to 5.5 mm, preferably 4.75 to 5.25 mm, preferably 5 mm. Further, each injection port 306 includes a sealing septum 308. In some embodiments, the injection port 306 may be defined by or formed by the sealing septum 308. The injection port can be formed in or defined by the sealing septum. For example, the injection port can be the entirety of a flat, uniformly thick sealing septum that covers an entirety of the circular aperture. In some embodiments, the injection port is defined in a central portion of the sealing septum. Such a central portion of the sealing septum can be concentric with the concentric circular aperture. The sealing septum can be configured to receive or be pierced by a sharp, hollow instrument, such as a needle. This can allow for transmitting components in and out of the plurality of wells 204 via the injection port 306. For example, a hypodermic syringe equipped with a sharp, hollow, medical grade needle can pierce the sealing septum in the region of the injection port, pass through the concentric circular aperture, and access the well disposed below. In general, the sealing septum refers to a self-sealing structure formed of an elastomeric material. Examples of suitable elastomeric materials include but are not limited to rubbers such as natural rubber, nitrile rubber, butyl rubber, styrene-butadiene rubber, EPDM rubber, chloroprene, Neoprene®, hydrogenated nitrile rubber, and the like, silicones such as dimethylsilicone, methyl-phenylsilicone, methylvinylsilicone, mixtures thereof, and the like, fluoroelastomers such as FKM, FFKM, FEPM, and the like. In some embodiments, the elastomeric material is a rubber.

FIG. 3C and FIG. 3D depict a sealing septum 308 according to an exemplary embodiment of the present disclosure. The depicted sealing septum has a hollow frustoconical shape. The exemplary frustoconical shape of the exemplary sealing septum 308 includes a narrow end 310, a wide end 312 and a sloping wall 314 circumferentially connecting an edge of the narrow end 310 and an edge of the wide end 312. The injection port 306 is depicted within a small circular area in the center of the sealing septum. In general, the frustoconical shape describes a shape that is a frustum of a cone. A frustum of a cone is a geometric shape that is obtained by cutting off the top of a cone with a plane parallel to a base of the cone. The resulting shape has two parallel bases, one smaller than the other, connected by a curved lateral surface. In some embodiments, both the edge of the narrow end 310 and the edge of the wide end 312 are circular. It should be understood that either or both of the narrow end and wide end can be any suitable shape as described above. Preferably, the narrow and wide end each have a shape that matches a shape of the concentric aperture. In some embodiments, the narrow end 310 may have a diameter of around 7 mm. In some embodiments, the wide end 312 may a diameter of around 13 mm. In some embodiments, the sealing septum may have a shape which is substantially cylindrical. That is, the wide end and narrow end may have the same diameter. In the exemplary sealing septum depicted in FIG. 3C and FIG. 3D, the sealing septum has a height ‘H2’ measured perpendicularly from a plane defined by the edge of the narrow end 310 to a plane defined by the edge of the wide end 312. In some embodiments, the height ‘H2’ is less than the diameter of the narrow end 310. In some embodiments, the height ‘H2’ is greater than the diameter of the narrow end 310. In some embodiments, the sealing septum has the injection port disposed or formed in a center of the sealing septum. In some embodiments, a thickness of the injection port is less than a height ‘H2’ of the sealing septum. That is, the injection port is formed by a thinned portion of the sealing septum. In some embodiments, the thinned portion of the sealing septum has an upper thinned portion surface that is substantially coplanar with an upper surface of the sealing septum. In some embodiments, the thinned portion of the sealing septum has a lower thinned portion surface that is not substantially coplanar with a lower surface of the sealing septum. This thinned portion may be advantageous for being easier to pierce with a needle.

In some embodiments, the sealing septum has a height ‘H2’ such that a lower surface of the sealing septum does not contact the base. That is, the sealing septum does not create a seal about the well. In some embodiments, there is a shared headspace above the plurality of wells such that gas exchange can occur between the wells. That is, a sealing septum does not create a seal around the corresponding well such that the well is completely isolated from other wells in the plate.

In some embodiments, the sealing septum may have a retention groove disposed about a periphery of the sealing septum in a region configured to be placed within the concentric aperture. In some embodiments, the retention groove can have a diameter equal to the diameter of the concentric aperture. For example, the frustoconical shape described of the sealing septum can have a portion immediately above the lid that has a diameter greater than the concentric aperture and a portion immediately below the lid that has a diameter greater than the concentric aperture. This retention groove may be advantageous for securing the sealing septum in the concentric aperture.

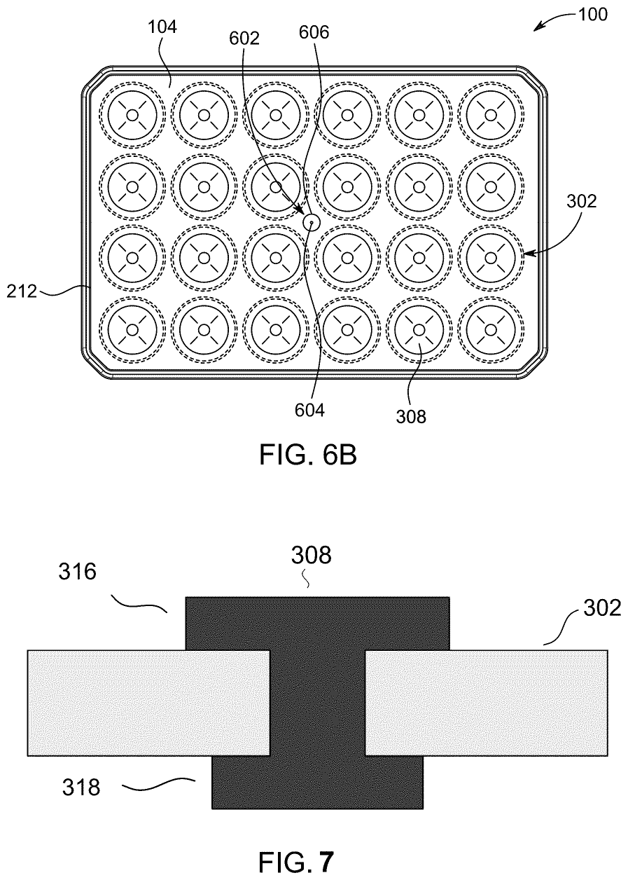

In some embodiments, the sealing septum can have an upper retention flange disposed about an upper end (e.g., the wide end). The upper retention flange can have a diameter greater than a body of the sealing septum. The upper retention flange can be configured to be in contact with an upper surface of the lid in an area immediately surrounding the concentric aperture. The upper retention flange may be useful for preventing the sealing septum from being pushed into the concentric aperture or for otherwise retaining the sealing septum in the concentric aperture. In some embodiments, the sealing septum can have a lower retention flange disposed about a portion of the sealing septum immediately below the circular aperture. In some embodiments, the lower retention flange is disposed about the narrow end of the sealing septum. The lower retention flange can have a diameter greater than a body of the sealing septum. The lower retention flange can be configured to be in contact with an underside of the lid in an area immediately surrounding the concentric aperture. The lower retention flange may be useful for preventing the sealing septum from being pulled out of the concentric aperture or for otherwise retaining the sealing septum in the concentric aperture. An exemplary sealing septum 308 having both an upper retention flange 316 and a lower retention flange 318 is shown in FIG. 7. This exemplary sealing septum 308 has a cross-sectional shape similar to a capital letter I. In some embodiments, the circular structure has an upper retention ridge disposed on an upper surface of the lid. In some embodiments, the upper retention ridge is configured to be disposed immediately outside of the upper retention flange. The upper retention ridge may be advantageous to prevent lateral movement of the sealing septum. In some embodiments, the circular structure has a lower retention ridge disposed on a lower surface (i.e., underside) of the lid. In some embodiments, the lower retention ridge is configured to be disposed immediately outside of the lower retention flange. The lower retention ridge may be advantageous to prevent lateral movement of the sealing septum. In some embodiments, the sealing septum has an upper retention flange having a diameter of 8.5 to 18.5 mm, preferably 9.5 to 17.5 mm, preferably 10.5 to 16.5 mm, preferably 11 to 15.5 mm, preferably 11.5 to 14.5 mm, preferably 12 to 14 mm, preferably 12.5 to 13.5 mm, preferably 12.75 to 13.25 mm, preferably 13 mm.

The sealing septum 308 is configured to be detachably inserted into the concentric circular aperture 304. In some embodiments, an upper surface of the sealing septum protrudes above an upper surface of the lid. That is, each sealing septum forms a raised protrusion or bump on the lid in the area of the corresponding well. In some embodiments, the sealing septum 308 provides an atmospherically sealed environments to the plurality of wells 204 of the multi-well plate 100 while allowing for access to the contents of the well via needle or other suitable sharp instrument. In some embodiments, the sealing septum 308 is manufactured with an interference fit with the concentric circular aperture 304, thereby resulting in an air-tight joint.

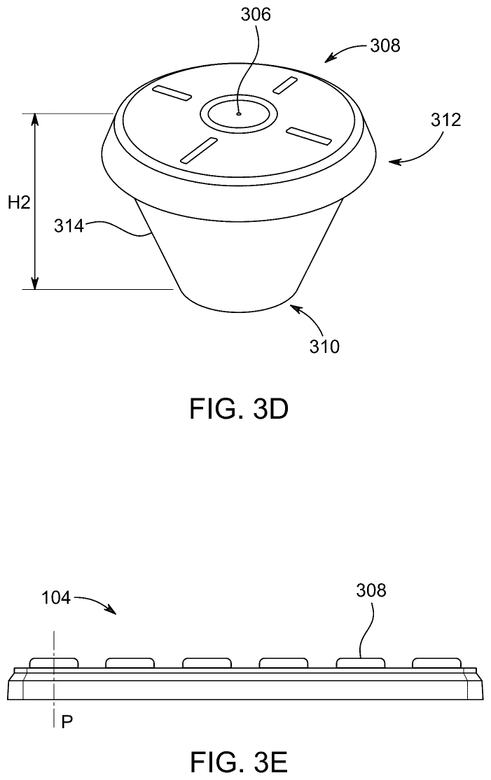

Referring to FIG. 4A, a schematic exploded view of an exemplary multi-well plate 100 is illustrated, according to certain embodiments. In the exemplary embodiment depicted in FIG. 4A, the lid 104 including the plurality of circular structures 302 is detached from the base 102 including the gasket 216 and the plurality of wells 204 is shown. During an assembly of the lid 104 with the base 102, the gasket 216 is attached to the perimeter wall 210 of the base 102. Further, the lid 104 and the base 102 are configured to be aligned in such a way that the central axis ‘C’ of each well of the plurality of wells 204 aligns with the central axis ‘P’ of each circular structure of the plurality of circular structures 302. Further, the alignment assures secure and appropriate assembly of the lid 104 with the base 102.

Referring to FIG. 4B, a schematic side view of an assembled multi-well plate 100 including the base 102 and the lid 104 is illustrated, according to an exemplary embodiment. As can be seen from FIG. 4B, in this exemplary embodiment, the lid 104 is configured to rest on the gasket 216 attached to the perimeter wall 210 and thereby form a seal about a periphery of the rectangular array of the multi-well plate 100. The seal formed about the periphery of the rectangular array prevents mixing of the internal atmosphere defined within the base 102 and the lid 104 with the external atmosphere. As such, the components present in the plurality of wells 204 are prevented from exposing to the external atmosphere, which in turn, may contaminate the components present in the plurality of wells 204 of the multi-well plate 100. The lid 104 is further configured to rest on the gasket 216 such that each circular structure of the plurality of circular structures 302 does not prevent gas exchange between a well and another well, of the plurality of wells 204. The sealing septum 308 along with the injection port 306 of each of the plurality of circular structures 302 is configured to be detachably coupled with the plurality of wells 204 of the base 102. As mentioned above, the plurality of wells 204 are arranged in the rectangular array, therefore, the plurality of circular structures 302 are consequently arranged in the rectangular array, alternatively referred to as the regular rectangular grid.

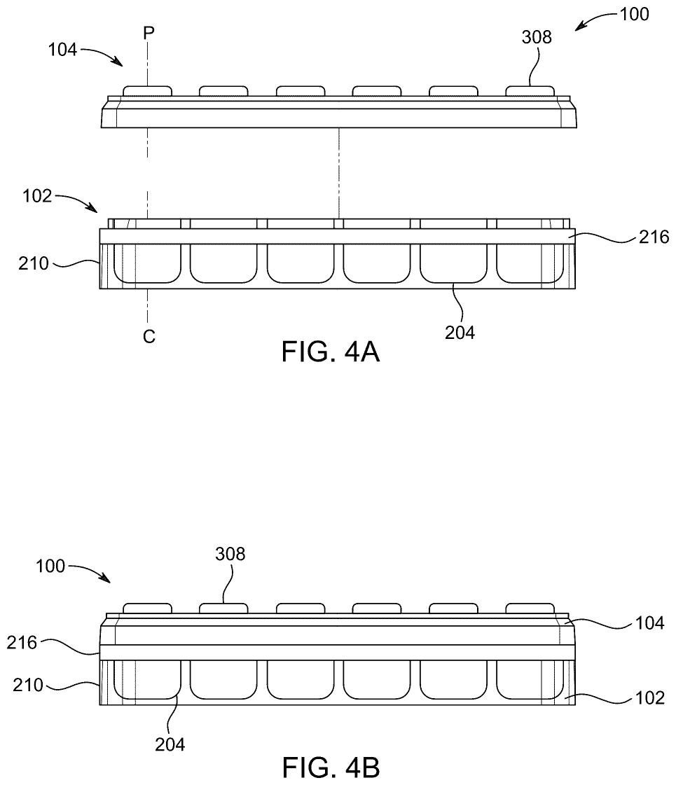

In some embodiments, the multi-well plate 100 includes a lid retention device. Referring to FIG. 5, a schematic side view of the multi-well plate 100 showing a lid retention device 502 is illustrated, according to an exemplary embodiment. In general, the lid retention device can be any suitable device configured to securely attach the lid 104 to the base 102. In some embodiments, the base 102 of the multi-well plate 100 includes the lid retention device 502. In some embodiments, the lid 104 of the multi-well plate 100 includes the lid retention device 502. In some embodiments, the lid retention device 502 includes structures disposed on both the base and the lid. For example, the lid retention device can be or include a first structure disposed on the base configured to interact with a corresponding second structure disponed on the lid. In some embodiments, the lid retention device 502 may be attached to two opposite sides of the base 102 to securely attach the lid 104 thereto. In some embodiments, the lid 104 may be fixedly and rotatably attached or hinged to the base 102 at one side and the opposite side may be provided with the lid retention device 502. In another embodiment, the lid 104 may be detachably and rotatably attached or hinged to the base 102 at one side and the opposite side may be provided with the lid retention device 502. Examples of lid retention devices include, but are not limited to snap-fit mechanisms, clips, buckles, latches, bolts, screws, fasteners, magnetic attachment devices, and the like. In some embodiments, the lid retention device 502 may include a latching system, a locking system, or a combination thereof. The latching system may include a toggle latch, a push-to-close latch, a sliding latch, a compression latch, and the like.

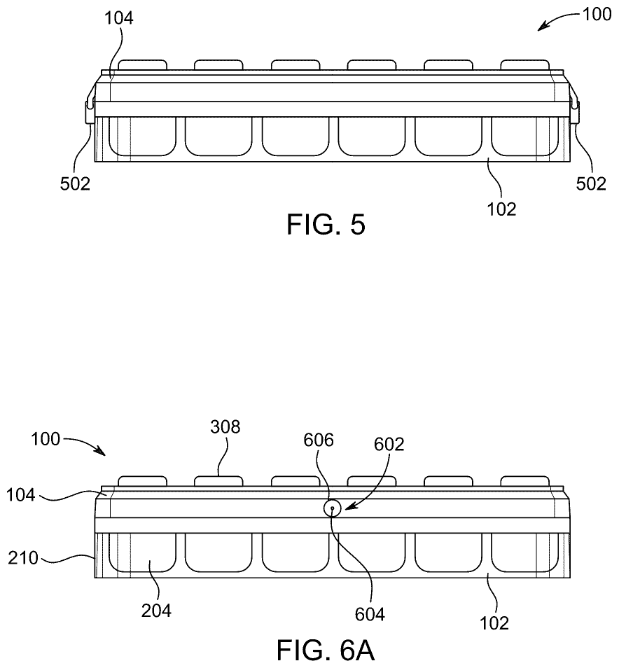

In some embodiments, the multi-well plate includes a gas inlet. Referring to FIG. 6A, a schematic side view of the multi-well plate 100 showing a gas inlet 602 disposed on a side of the lid 104 is illustrated, according to an exemplary embodiment. In particular, the gas inlet 602 includes an injection port 604 a sealing septum 606 defining the injection port 604. In general, the sealing septum can be a sealing septum as described above. The gas inlet 602 may configured to allow for introduction of one or more gases into the multi-well plate 100. In other words, the gas inlet 602 may be configured to be in fluid communication with the plurality of wells 204 or a headspace above the plurality of wells and the one or more gases may be transmitted through the gas inlet 602 to enforce a particular gaseous environment inside the multi-well plate 100. In a non-limiting example, a particular cell culture present in the plurality of wells 204 may need a carbon dioxide environment for proliferation and growth thereof. Herein, the gas inlet 602 may be used to transmit carbon dioxide in order to provide required gaseous environment. The injection port 604 and the sealing septum 606, included in the gas inlet 602, provide a self-sealing orifice for the transmission of the one or more gases to the multi-well plate 100. In particular, the sealing septum 606, while transmission of the one or more gases, prevents atmospheric contamination of the components present inside the plurality of wells 204 of the multi-well plate 100.

Referring to FIG. 6B, a schematic top view of the multi-well plate 100 showing the gas inlet 602 disposed on a top of the lid 104 is illustrated, according to certain embodiments. As such, the gas inlet 602 disposed on the top of the lid 104 is not disposed above a corresponding well of the plurality of wells 204. In some embodiments, the gas inlet 602 is configured to be positioned centrally on the top of the lid 104. In particular, the gas inlet 602 is not directly over any well of the plurality of wells 204. The positioning of the gas inlet 602 on the top of the lid 104 allows for an even distribution of the one or more gases transmitted therethrough and prohibits partial distribution of the one or more gases to a particular well of the plurality of wells 204 and thereby creating the required gaseous environment inside the multi-well plate 100. In some embodiments, the constructional details, and dimensional specifications of the gas inlet 602 disposed on the top of the lid 104 is similar to the gas inlet 602 disposed on the side of the lid 104. Further, the gas inlet 602 disposed on the top of the lid 104 also includes the injection port 604 and the sealing septum 606, where the injection port 604 and sealing septum 606 perform similar functions as described above.

Aspects of the present disclosure describe a multi-well plate 100 with ability to isolate a plurality of cell-cultures from surrounding environments and possible contaminants. The multi-well plate 100, in conjunction with the lid 104 allows for manipulation of the plurality of cell-cultures, without compromising on the sanitation and integrity of the plurality of cell cultures, present in the plurality of wells 204 of the multi-well plate 100. The multi-well plate 100 may be employed in conventional incubators that are shared property in a majority of laboratories. The sealing septum 308 in the lid 104 prevents the risk of contamination of the plurality of cell cultures from fungi, mold, mildew, external bacteria, external viruses, and other biological contaminants. The multi-well plate 100 as described in the present disclosure further allows for a targeted atmosphere manipulation inside the multi-well plate 100 without prior atmospheric conditions affecting the plurality of cell cultures present in the multi-well plate 100. The multi-well plate 100 of the present disclosure may eliminate a need of a plurality of multi-well plates for different cell-cultures, by providing a segregated and sterile proliferation environment to the plurality of cell cultures present inside the multi-well plate. The above-mentioned reduction in number of multi-well plates may eventually result in decreased environmental impact from plastics of multi-well plates and further improve economical aspects of a particular use case of the multi-well plate 100.

Numerous modifications and variations of the present disclosure are possible in light of the above teachings. It is therefore to be understood that within the scope of the appended claims, the invention may be practiced otherwise than as specifically described herein.

1. A multi-well plate, comprising,
a base comprising,
a plurality of wells arranged in a rectangular array, each well having a cylindrical shape with a circular opening on a top end of the well,
a perimeter wall arranged about a periphery of the rectangular array and including a stacked edge arranged about an entirety of the perimeter wall, and
a gasket attached circumferentially to the perimeter wall at a notch on the stacked edge of the perimeter wall; and
a lid comprising a plurality of circular structures axially aligned with the plurality of wells of the base, each circular structure including;
a concentric circular aperture and
an injection port disposed at a center of each concentric circular aperture on each of the plurality of circular structures forming a plurality of injection ports, each injection port comprising a sealing septum,
wherein the lid is configured to rest on the gasket attached circumferentially to the perimeter wall and thereby forming a seal about a periphery of the rectangular array.

2. The multi-well plate of claim 1, wherein the seal formed about the periphery of the rectangular array prevents mixing of an internal atmosphere defined within the base and the lid with an external atmosphere.

3. The multi-well plate of claim 1, wherein each well in the rectangular array has a corresponding circular projection in the lid.

4. The multi-well plate of claim 1, wherein the lid is configured to rest on the gasket such that a circular structure of the plurality of circular structures does not prevent gas exchange between a well and another well.

5. The multi-well plate of claim 1, wherein the plurality of circular structures is arranged to form a regular rectangular grid.

6. The multi-well plate of claim 1, wherein
each sealing septum of the lid is formed of an elastomeric material and has a hollow frustoconical shape with
a narrow end,
a wide end, and
a sloping wall circumferentially connecting an edge of the narrow end and an edge of the wide end; wherein
both the edge of the narrow end and the edge of the wide end are circular;
the narrow end is closed, and the wide end is open; and
a height of the sealing septum measured perpendicularly from a plane defined by the narrow end to a plane defined by the edge of the wide end is less than a diameter of the narrow end.

7. The multi-well plate of claim 6, wherein the elastomeric material is a rubber.

8. The multi-well plate of claim 1, wherein the gasket is formed of an elastomeric material.

9. The multi-well plate of claim 8, wherein the elastomeric material is a rubber.

10. The multi-well plate of claim 1, wherein a ratio of a diameter of the circular opening on the top end of a well to a diameter of the concentric circular aperture is 1:1 to 10:1.

11. The multi-well plate of claim 1, further comprising a gas inlet disposed on the lid and comprising an injection port comprising a sealing septum.

12. The multi-well plate of claim 11, wherein the gas inlet is disposed on a top of the lid.

13. The multi-well plate of claim 12, wherein the gas inlet is disposed on a top of the lid such that the gas inlet is not disposed above a corresponding well of the plurality of wells.

14. The multi-well plate of claim 11, wherein the injection port of the gas inlet is formed of an elastomeric material.

15. The multi-well plate of claim 1, further comprising a lid retention device configured to securely attach the lid to the base.

16. The multi-well plate of claim 1, wherein the gasket has a width of 1.0 to 2.5 mm.

17. The multi-well plate of claim 1, wherein the wells are arranged into an array having a width to length ratio of 2:3.

18. The multi-well plate of claim 1, wherein each well has a diameter of 15 to 22.5 mm.

19. The multi-well plate of claim 1, wherein each concentric circular aperture has a diameter of 2.5 to 15 mm.

20. The multi-well plate of claim 1, wherein the sealing septum has an upper retention flange having a diameter of 8.5 to 18.5 mm.
